(19) United States
(12) Patent Application Publication
(10) Pub. No.: US 2024/0261361 A1
(43) Pub. Date: Aug. 8, 2024

(54) ANTI-ALLERGENIC OCULAR COMPOSITIONS

(71) Applicant: OCuSOFT, Inc., Rosenberg, TX (US)

(72) Inventors: Nat Adkins, JR., Richmond, TX (US); Paramita Sarkar, Richmond, TX (US); Troy Smith, Richmond, TX (US)

(21) Appl. No.: 18/639,128

(22) Filed: Apr. 18, 2024

Related U.S. Application Data
(63) Continuation-in-part of application No. 17/213,986, filed on Mar. 26, 2021.
Application No. 17/213,986 is a continuation-in-part of application No. 16/508,626, filed on Jul. 11, 2019, now Pat. No. 10,959,940.
(60) Provisional application No. 63/497,369, filed on Apr. 20, 2023.
(60) Provisional application No. 62/697,213, filed on Jul. 12, 2018.

(51) Int. Cl. A61K 36/82; A61K 36/61; A61K 47/10; A61K 47/18; A61K 47/26

(52) U.S. Cl. CPC A61K 36/82; A61K 36/61; A61K 47/10; A61K 47/18; A61K 47/26

(57) ABSTRACT

An anti-allergenic product having: at least 0.01% w/w of a plant extract, wherein the plant extract is selected from the group consisting of Camellia sinensis leaf extract, Melaleuca alternifolia leaf oil, and mixtures thereof; an amphiphilic preservative, and a glycolipid. The eyelid cleansing composition is configured to relieve redness, discomfort and irritation in and around the eyelid.

### CROSS REFERENCE TO RELATED APPLICATIONS

This application claims priority to U.S. Provisional Patent Application Ser. No. 63/497,369 filed on Apr. 20, 2023. This application is a continuation-in-part of U.S. patent application Ser. No. 17/213,986 filed on Mar. 26, 2021 which is a continuation-in-part of U.S. Ser. No. 16/508,626 filed on Jul. 11, 2019 which claims the benefit of Provisional U.S. Patent Application No. 62/697,213, filed Jul. 12, 2018, the entire contents and disclosures of all of which, both express and implied, are incorporated herein by reference.

### FIELD OF THE INVENTION

The invention relates generally to an anti-allergenic product for alleviating the symptoms associated with eyelid allergy.

### BACKGROUND

Ocular health refers to eyes as well as structures associated with the eyes, eyelids for example. The eyelids are important in overall ocular health because they protect the eyes from dangers such as approaching objects or from airborne contaminants, such as pollen, dust particles or other foreign bodies. The eyelids contain essential glands; the lacrimal glands and meibomian glands that produce layers of tear film that are critical for healthy eyes. When an individual blinks, a new tear film is created, and tears are distributed across the cornea to lubricate the surface of the eye. This blinking action also “flushes” foreign materials from the eye.

The eyelids, however, are subject to allergies and bacterial infection of the surface of the skin. Seasonal allergies can also cause red, itchy, irritated eyes and eyelids. Allergy eye drops relieve eye symptoms but do not calm irritated eyelids. Therefore, there is a need for a product that can alleviate the symptoms associated with allergic conditions of the eyelids.

### SUMMARY

According to an embodiment, an anti-allergenic composition comprises at least 0.01% w/w of a plant extract, wherein the plant extract is selected form the group consisting of Camellia sinesis leaf extract, Malaleuca alternifolia leaf oil, and mixtures thereof. The composition further comprises a glycolipid, an amphiphilic preservative and water. In an embodiment, the glycolipid is a sphingolipid selected from the group consisting of sphingolipids, ceramides, glycerophospholipids, sphingolipid derivatives, and combinations thereof.

In an exemplary embodiment, a sphingolipid is selected from the group consisting of sphingosine, sphinganine, phytosphingosine, a phytosphingosine salt, tetraacetyl-phytosphingosine, N-acetylphytosphingosine, and mixtures thereof. The sphingolipid is preferably in the range of 0.1% to 0.3% w/w.

In one or more embodiments, the preservative is present in the range of 0.01% to 5% w/w. In a specific embodiment, the preservative is lauramine oxide.

According to an embodiment, the anti-allergenic composition additionally comprises at least one polyol, wherein the polyol has 2-12 carbon atoms. The at least one polyol can be a 1,2-diol selected from the group consisting of 1,2-hexanediol, 1,2-octanediol, 1,2-decanediol, and mixtures thereof. Embodiments of the composition comprise at least one polyol in the range of 0.01% to 5% w/w.

Exemplary embodiments of the anti-allergenic composition further comprise one or more surfactants present in the range of 0.1 to 20% w/w. Of the one or more surfactants, a first surfactant has an HLB value between 10 and 18, and a second surfactant, if present, has an HLB value between 10 and 18 or 5 and 10. In a specific embodiment, the surfactants comprise decyl glucoside and polysorbate 20.

In an embodiment, the anti-allergenic composition further comprises a foam stabilizer. The foam stabilizer is, preferably, a polyethylene glycol diester of methyl glucose and a fatty acid.

In certain embodiments of the anti-allergenic composition, the pH is in the range of 3.5 to 6.5, the refractive index is about 1.33 to 1.35, and the specific gravity is about 0.99 to 1.01.

Additional embodiments of the anti-allergenic composition comprise one or more moisturizers, emollients, humectants, and lubricants and one or more skin conditioning agents, antioxidants, soothing agents, cooling agents, viscosity modifiers, and nutrients.

According to an embodiment, the anti-allergenic composition is combined with a substrate capable of being impregnated with a desired amount of the composition and, wherein the composition-impregnated substrate is configured as a single use product.

In an exemplary embodiment, the anti-allergenic composition consists of 0.1-0.3% phytosphingosine HCl, 0.01-5% hexanediol, 0.01-5% octanediol, 0.1-20% polysorbate 20, 0.1-20% decyl glucoside, at least 0.01% tea tree oil, at least 0.01% green tea extract, 0.01-5% lauramine oxide, and water to 100%. The pH is in the range of 3.5 to 6.5, the refractive index is about 1.33 to 1.35, and the specific gravity is about 0.99 to 1.0.

In an embodiment, a method for treating eyelid allergy comprises gently applying the anti-allergenic composition to the eyelid surface and surrounding ocular area to alleviate redness, discomfort, and irritation in the area of application. The composition can be configured as a leave-on or rinse-off formulation.

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the anti-allergenic composition, unless otherwise specified. Additionally, all composition percentages are based on totals equal to 100 wt. %, unless otherwise specified.

The products and methods described herein can “comprise,” “consist essentially of,” or “consist of” any of the ingredients or steps disclosed throughout the specification. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients/steps of the present invention and do not exclude other ingredients or steps described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients/steps in addition to those recited in the claim, but only if the additional ingredients do not materially alter the basic and novel characteristics of the claimed invention. Generally, additives may not be present at all or only in trace amounts. However, it may be possible to include up to about 10% by weight of materials that could materially alter the basic and novel characteristics of the invention as long as the utility of the composition (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. In one non-limiting embodiment the terms are defined to be within 1%, more preferably within 0.1%, and most preferably within 0.01%. The term “substantially” and its variations are defined as being largely but not necessarily wholly what is specified as understood by one of ordinary skill in the art.

As used herein, the term “effective amount” of a composition refers to an amount sufficient to elicit the desired biological response. As will be appreciated by those of ordinary skill in this art, and effective amount of a substance may vary depending on such factors as the desired biological endpoint, the patient, etc. In some embodiments, a therapeutically effective amount of a composition is an amount that is sufficient, when administered to a subject suffering from or susceptible to a disease, disorder, and/or condition, to treat, inhibit, reduce, prevent, and/or delay the onset of one or more symptoms of eyelid allergy. The terms “treat” or “reduce” or any variation of these terms includes a qualitative or quantitative decrease in allergy symptoms. For example, the effective amount/therapeutically effective amount of the composition to treat eyelid allergy is the amount that alleviates, ameliorates, relieves, inhibits, prevents, delays onset of, reduces severity of and/or reduces incidence of one or more symptoms or features associated with allergic conditions of the eyelids.

The anti-allergenic composition can include anti-inflammatory, anti-microbial, anti-fungal and anti-irritant agents. In one embodiment, the composition is an eyelid cleanser for removing pollen, contaminants and allergens from the eyelids and eyelid margins/arcas surrounding the eyes/eyelids. The composition can be configured to prevent or reduce eyelid allergy-like symptoms, namely, redness, itching, roughness, or inflammation of the eyelids or its surrounding areas. The composition has an effective amount of: one or more plant extracts, a lipophilic or amphiphilic agent, a preservative with anti-microbial activity, and a suitable solvent or carrier.

The plant extracts can be selected from a group consisting of Camellia Sinensis (green tea) leaf extract, Melaleuca alternifolia (tea tree) leaf oil, Citrus sinensis, chamomile flowers, and combinations thereof. In one specific embodiment, the plant extract can include a mixture of green tea leaf extract and tea tree leaf oil. The selected plant extracts will contain an effective amount (i.e., at least a 0.01% w/w of the composition) of a polyphenol and a terpene compound. Such plant extracts typically have anti-microbial, anti-fungal and/or anti-inflammatory activity.

For example, certain Camellia Sinensis or green tea leaf extracts have been shown to contain a polyphenol. The polyphenol can include a flavanol derivate. Flavanol derivatives can include catechin, epigallocatechin and epigallocatechin gallate. Green tea leaf extracts generally have an antioxidant effect that can protect the skin from the damaging effect of free radicals. In addition, certain green tea extracts contain an anti-inflammatory agent that reduces inflammation in the skin. Some green tea extracts have also been shown to have a moisturizing effect and to serve as an inhibitor of epidermal collagenase (a collagen-reducing enzyme that breaks down collagen) to assist in maintaining a firm and elastic skin.

Tea tree leaf oil or tea tree oil can generally be obtained by steam distillation from the Australian native plant Melaleuca alternifolia, and contains around a hundred components, such as, terpinenes, terpenes, and related derivatives and combinations. More specifically, tea tree oil often contains monoterpenes, sesquiterpenes and related alcohols.

One important aspect of the composition is related to its anti-microbial activity typically rendered by one or more anti-microbial agents. In an embodiment, the composition includes a glycolipid. The glycolipid can be selected from the group consisting of sphingolipids, ceramides, glycerophospholipids, sphingolipid derivative and combinations thereof. In an embodiment, a sphingoid base, selected from a group consisting of sphingosine, sphinganine, phytosphingosine, a phytosphingosine salt, tetraacetyl-phytosphingosine, N-acetylphytosphingosine and mixtures thereof is selected. In one specific embodiment, the sphingoid base includes about 0.1% to about 0.3% phytosphingosine and/or phytosphingosine hydrochloride (HCl) as an active ingredient. In one embodiment, the composition includes about 0.2% Phytosphingosine HCl. Phytosphingosine and Phytosphingosine HCl can inhibit the growth of microorganisms on the skin, reduce redness and inflamed skin and are active at very low concentrations. PSG HCl can contribute to the prevention of loss of moisture from the skin and boosting collagen synthesis while reducing its degradation, and soothing inflamed skin. It can also exhibit skin protectant and anti-inflammatory properties at concentrations as low as 0.1%.

Preferred embodiments of the composition comprise a preservative having an effective anti-microbial activity. One such preservative with significant anti-microbial properties is lauramine oxide. Lauramine oxide is an ideal amphiphilic preservative for the ocular region, as it is compatible with a wide range of surfactant systems including anionic, cationic, amphoteric, nonionic systems, and their various combinations. In one or more embodiments, the preservative is selected from the group consisting of lauramine oxide, myristamine oxide, a combination of lauramine oxide and myristamine oxide, N,N-dialkyl amine oxide with long alkyl chain between C10-C18, and mixtures thereof. In an embodiment, the N,N-dialkyl is preferably methyl. And, the N,N-dialkyl amine oxide with long alkyl chain between C10-C18 is preferably oleyldimethylamine oxide.

The composition further includes a polyol having 2-12 carbon atoms. Preferably, at least one 1,2-diol compound is included for solubilization of the glycolipid. The 1,2-diol can be included in the composition for its anti-microbial enhancing activity. Preferred 1,2-diols are selected from the group consisting of 1,2-hexanediol is a synthetic preservative with a broad spectrum anti-microbial activity. Similarly, 1,2-octanediol (also known as caprylyl glycol) is another active substance which functions as a preservative and anti-microbial.

A blend of one or more surfactants may also be included in the anti-allergenic composition to facilitate solubilization of tea tree oil. Various surfactants may be included in the composition to achieve a desired foaming capacity and/or cleansing capability. The inclusion of a surfactant will enhance the ability of the composition to dissolve and remove oil, antigens, debris and desquamated skin. Preferable surfactants have a foaming ability and include nonionic or amphoteric surfactants having a HLB value between 10 and 18. If more than one surfactant is used, then at least one surfactant should have an HLB value between 10 and 18 and the other surfactant(s) should either have an HLB between 10 and 18 or an HLB value between 5 and 10.

Suitable surfactants can include amphoteric surfactants, anionic surfactants, and nonionic surfactants. Suitable amphoteric surfactants include, but are not limited to alkyldimethyl betaines, alkylamido betaines, sulfobetaines, and imidazoline amphoterics. Suitable anionic surfactants include, but are not limited to fatty alcohol sulfates, alpha olein sulfonates, sulfosuccinates, sarcosinates, phosphate esters, and carboxylates. Suitable nonionic surfactants include, but are not limited to alkanolamides, ethoxylated amides, esters, alkylated alcohols, alkylpolyglucosides, amine oxides, sorbitan esters, and ethoxylates.

The one or more surfactants may include, but are not limited to, sorbitan esters (e.g. Span 20 or sorbitan monolaurate), polyethylene glycol (PEG) modified surfactants (e.g. polysorbate 20, Brij 52, PEG-75 Lanolin), modified phospholipids, modified sugars (e.g., alkyl polyglucosides, alkyl glucosides, fatty acid glucamides), amineoxides, and/or block copolymers such as Pluronics (Pluronic F120) or tyloxapol. The total concentration of surfactants will be in the range of 0.1 to 20% w/w of the final formulation but, more preferably in the 0.5-5% range. The inclusion of a surfactant can increase the cleansing ability of the composition and provide it with a foaming capability. Foams are considered to have optimal cleansing capability.

A preferred surfactant system of the composition comprises decyl glucoside and polysorbate 20 to facilitate solubilization of tea tree oil. The system is optimized to enhance the foaming characteristics of the composition and the delivery of active ingredients to the skin surface. The HLB values of decyl glucoside and polysorbate 20 are 12.8 and 16.7, respectively.

Although optional, a foam stabilizer may also be added to the composition, and may include, without limitation, a polyethylene glycol diester of methyl glucose and a fatty acid. The fatty acid can be selected from a group consisting of oleic acid, stearic acid, lauric acid, caprylic acid, and capric acid. Preferably, the one or more foam stabilizers includes PEG-120 methyl glucose dioleate.

One or more moisturizers, emollients, humectants, or lubricants are also commonly added to the composition and are generally referred to herein as moisturizers. Suitable moisturizers are propylene glycol, glycerin, polyethylene glycol (e.g. PEG 300, 400, 600) or generally, liquid polyols, nut oils and derivatives, rose water (floral extracts), cucumber extract, fruit extracts, sodium alginate, hyaluronic acid, diglyceride, triglyceride, PEG-75 lanolin, mineral oil, and silicone oil.

Additional optional components may also be added to the composition in amounts up to 5% w/w of the composition. These optional components include such compounds as skin conditioning agents (e.g., D-panthenol), antioxidants (e.g., Vitamin E and its derivatives, green tea extract, Vitamin C and its derivatives), anti-irritating/soothing agents (e.g., allantoin, aloe vera, tea tree oil), cooling agents (e.g., sorbitol, xylitol, menthol, thymol), viscosity modifiers (e.g., carboxymethycellulose, hypromellose, carbopol), and nutrients (e.g., minerals, vitamins, amino acids or polypeptides).

An exemplary embodiment of the composition is illustrated in Table 1. The embodiment has a pH in the range of 3.5 to 6.5, a refractive index of about 1.33 to 1.35, and a specific gravity of about 0.99 to 1.01. Skilled practitioners in the art will recognize that the ranges of the properties in the composition can be adjusted while maintaining physiological compatibility with the ocular region. The given ranges reflect an optimum for the following list of ingredients and are non-limiting of all possible embodiments of the present invention.

TABLE 1

| Ingredient | Purpose | % wt/wt |
|---|---|---|
| Phytosphingosine HCl | Skin protectant/Anti- | 0.1-0.3% |
| | inflammatory | |
| Hexanediol | Antimicrobial | 0.01-5% |
| Octanediol | Antimicrobial | 0.01-5% |
| Polysorbate 20 | Surfactant | 0.1-20% |
| Decyl glucoside | Surfactant | 0.1-20% |
| Tea Tree Oil | Plant extract | >0.01% |
| Green Tea Extract | Plant extract | >0.01% |
| Lauramine oxide | Preservative | 0.01-5% |
| Purified water | Vehicle | q.s. to 100% |

The composition is substantially non-irritating to the eye. Advantageously, the composition does not contain astringents such as zinc or zinc salts.

The composition can be selected from a group consisting of a solution, a mist, a foam, a gel, a spray, a lotion, a suspension, an emulsion, an ointment and combinations thereof.

In one embodiment, the composition may be combined with a suitable substrate to form an anti-allergenic product for cleansing the eyelids. The substrate can be impregnated with a clinically effective amount of the composition. The substrate can be a wipe, a fabric pad or a towelette or any suitable material that is capable of absorbing the desired amount of the composition. The term “pad” refers to a thick piece of fabric that is capable of holding or absorbing the composition. The fabric can be a lint-free fabric, such as, rayon or another suitable material. In certain embodiments, the fabric pad may be a cotton pad. The fabric pad can comprise a textured surface.

The anti-allergenic product can be configured as a single use product. The anti-allergenic product can be provided in hermetically sealed package. Between 1-100 such packages can be provided in a container.

In another embodiment, a method for treating eyelid allergy involves providing the anti-allergenic product disclosed herein. The method further involves gently rubbing the product on or over an eyelid surface and/or an area surrounding the eyelid to relieve redness, discomfort and irritation in and around the eyelids. The composition can be configured as a non-residue forming composition. The composition can be left on the eyelid or rinsed off with water. The composition can clean and remove foreign material and debris from the application site including microorganisms, such as bacteria or allergens on the eyelids. It can also induce foaming, which assists in the cleansing ability of the formulation and prevent allergies and promote anti-inflammation and redness of the eyelids and eyelid margins.

The present invention can comprise or consist essentially of components as well as other components which are not recited (such as, biological gloves).

In one or more embodiments, the anti-allergenic ocular composition can be prepared by first preparing a glycolipid mixture. This involves adding predetermined amounts of 1,2-diols to a mixing vessel to facilitate solubilization of the glycolipid mixture. Antimicrobial glycolipid is then incorporated into the same vessel. The mixture is subsequently mixed at 500 RPM for 30 minutes or until the glycolipid is completely dissolved into a clear solution. Heating of the mixture is not required to fully dissolve the glycolipid.

In a separate vessel, about 90% of the final batch weight of purified water is added. Next, the dissolved glycolipid mixture is added to the purified water. The initial mixing vessel is rinsed, as needed, with purified water and the contents are transferred to the new vessel. The mixing process is initiated at 500 RPM.

While mixing, surfactants, plant extracts, preservatives, and any other additional ingredient described herein are added to the mixture. All containers and vessels are rinsed with purified water and transferred to the current mixing vessel. The mixing process is continued until homogeneity is achieved. Finally, the mixture is brought to the final weight with purified water and mixed for an additional 30 minutes to ensure homogeneity.

Ocular irritation potential was evaluated using EpiOcular, non-animal testing. The anti-allergenic formulation at 100% had an estimated Draize ocular irritation score of approximately “0”, with a “non-irritant” irritancy classification. Human Repeat Insult Patch Test was performed on a panel comprised of 50 human subjects. Crtically, no indication of a potential to elicit dermal irritation or sensitization by the anti-allergenic formulation was found.

Stability of the anti-allergenic composition was evaluated over 18 months at room temperature and 6 months at 40° C. Under both conditions, the pH, refractive index, and specific gravity of the composition remained unchanged from their initial values. Additionally, the composition maintained its clear to yellow appearance without any visible particulate matter forming throughout the study. Anti-microbial efficacy testing was performed on the 18-month, room temperature sample according to USP 51 and received a passing result.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. An anti-allergenic composition comprising:
(i) at least 0.01% w/w of a plant extract, wherein the plant extract is selected from the group consisting of Camellia sinensis leaf extract, Malaleuca alternifolia leaf oil, and mixtures thereof;
(ii) a glycolipid;
(iii) an amphiphilic preservative; and
(iv) water.

2. The anti-allergenic composition according to claim 1, wherein the glycolipid is selected from the group consisting of sphingolipids, ceramides, glycerophospholipids, sphingolipid derivatives, and combinations thereof.

3. The anti-allergenic composition according to claim 2, wherein the glycolipid is a sphingolipid selected from the group consisting of sphingosine, sphinganine, phytosphingosine, a phytosphingosine salt, tetraacetyl-phytosphingosine, N-acetylphytosphingosine, and mixtures thereof.

4. The anti-allergenic composition according to claim 3, wherein the sphingolipid is in the range of 0.1% to 0.3% w/w.

5. The anti-allergenic composition according to claim 1, wherein the amphiphilic preservative is present in the range of 0.01% to 5% w/w.

6. The anti-allergenic composition according to claim 1, wherein the amphiphilic preservative is selected from the group consisting of lauramine oxide, myristamine oxide, a combination of lauramine oxide and myristamine oxide, N,N-dialkyl amine oxide with long alkyl chain between C10-C18, and mixtures thereof.

7. The anti-allergenic composition according to claim 1, further comprising at least one polyol.

8. The anti-allergenic composition according to claim 7, wherein the polyol has 2-12 carbon atoms.

9. The anti-allergenic composition according to claim 8, wherein the polyol is a 1,2-diol selected from the group consisting of 1,2-hexanediol, 1,2-octanediol, 1,2-decanediol, and mixtures thereof.

10. The anti-allergenic composition according to claim 9, wherein the polyol is present in the range of 0.01% to 5% w/w.

11. The anti-allergenic composition according to claim 1, further comprising one or more surfactants.

12. The anti-allergenic composition according to claim 11, wherein the one or more surfactants are present in the range of 0.1 to 20% w/w.

13. The anti-allergenic composition according to claim 11, wherein the one or more surfactants comprise decyl glucoside and polysorbate 20.

14. The anti-allergenic composition according to claim 11, wherein a first surfactant has an HLB value between 10 and 18.

15. The anti-allergenic composition according to claim 14, wherein a second surfactant has an HLB value between 10 and 18 or between 5 and 10.

16. The anti-allergenic composition according to claim 1, further comprising a foam stabilizer.

17. The anti-allergenic composition according to claim 16, wherein the foam stabilizer is a polyethylene glycol diester of methyl glucose and a fatty acid.

18. The anti-allergenic composition according to claim 1, wherein the pH is in the range of 3.5 to 6.5.

19. The anti-allergenic composition according to claim 1, wherein the refractive index is about 1.33 to 1.35.

20. The anti-allergenic composition according to claim 1, wherein the specific gravity is about 0.99 to 1.01.

21. The anti-allergenic composition according to claim 1, further comprising one or more moisturizers, emollients, humectants, and lubricants.

22. The anti-allergenic composition according to claim 1, further comprising one or more skin conditioning agents, antioxidants, soothing agents, cooling agents, viscosity modifiers, and nutrients.

23. The anti-allergenic composition according to claim 1, wherein the composition is combined with a substrate capable of being impregnated with a desired amount of the composition.

24. The anti-allergenic composition and substrate according to claim 23, wherein the composition-impregnated substrate is configured as a single use product.

25. An anti-allergenic composition consisting of:
0.1-0.3% phytosphingosine HCl,
0.01-5% hexanediol,
0.01-5% octanediol,
0.1-20% polysorbate 20,
0.1-20% decyl glucoside,
at least 0.01% tea tree oil,
at least 0.01% green tea extract,
0.01-5% lauramine oxide, and
water q.s. to 100%.

26. A method for treating eyelid allergy, comprising:
providing the anti-allergenic composition according to claim 1; and
gently applying the composition to the eyelid surface and surrounding ocular area,
wherein the composition is configured to alleviate redness, discomfort, and
irritation in the area of application.

27. The method according to claim 26, wherein the anti-allergenic composition is configured as a leave-on or rinse-off composition.
